(19) United States
(12) Patent Application Publication
(10) Pub. No.: US 2010/0249782 A1
(43) Pub. Date: Sep. 30, 2010

(54) INTRAMEDULLARY NAIL TARGETING DEVICE

(76) Inventor: Alfred A. Durham, Roanoke, VA (US)

Correspondence Address: Intellectual Property Dept.;Dewitt Ross & Stevens SC, 2 East Mifflin Street, Suite 600, Madison, WI, 53703-2865, US

(21) Appl. No.: 12/763,604

(22) Filed: Apr. 20, 2010

Related U.S. Application Data
(63) Continuation-in-part of application No. 12/552,726, filed on Sep. 2, 2009.
Application No. 12/552,726 is a continuation-in-part of application No. 10/679,166, filed on Oct. 3, 2003, now Pat. No. 7,753,913.
(60) Provisional application No. 61/214,060, filed on Apr. 20, 2009.
(60) Provisional application No. 61/190,709, filed on Sep. 2, 2008.
(60) Provisional application No. 60/415,952, filed on Oct. 3, 2002.

(51) Int. Cl. A61B 17/58; A61B 17/56

(52) U.S. Cl. 606/62; 606/96

(57) ABSTRACT

An intramedullary nail targeting apparatus for targeting and drilling screw openings in the intramedullay nail is provided herein. A preferred version of the targeting apparatus includes a magnetic targeting device, a nail extension for connecting to an intramedullary nail, and a magnet member, preferably in a “bucking configuration,” for affixing to the intramedullary nail at a defined position relative to the screw openings in the nail. The nail extension includes a targeting arm with one or more bores which align with the screw openings in the nail when the targeting arm is aligned with the intramedullary nail. The magnetic targeting device includes a support member with a sensor array that extends through one of the bores on the targeting arm to target the magnet member, thereby aligning the targeting arm with the intramedullary nail. A second bore on the targeting arm can then be used for drilling through the bone at the position of an aligned screw opening. Methods for using the targeting apparatus for targeting and drilling screw openings in intramedullary nails or openings in bone plates are also described herein.

### CROSS-REFERENCE TO RELATED APPLICATIONS

This application claims priority under 35 USC §119(e) to U.S. Provisional Patent Application 61/214,060 filed Apr. 20, 2009, and is a continuation-in-part under 35 USC §120 of U.S. patent application Ser. No. 12/552,726 filed Sep. 2, 2009, which claims priority under 35 USC §119(e) to U.S. Provisional Patent Application 61/190,709 filed Sep. 2, 2008 and is a continuation-in-part under 35 USC §120 of U.S. patent application Ser. No. 10/679,166 filed Oct. 3, 2003, which claims priority under 35 U.S.C. §119(e) to U.S. Provisional Patent Application 60/415,952 filed Oct. 3, 2002, all of which are incorporated herein by reference in their entirety.

### FIELD OF THE INVENTION

The present invention is directed to a targeting device in general and specifically relates to an intramedullary nail targeting device and method for positioning locking screws for intramedullary nails.

### BACKGROUND

Devices for targeting of distal holes or openings in orthopedic hardware such as intramedullary nails include mechanical targeting devices and magnetic targeting devices.

Examples of conventional mechanical targeting devices for intramedullary nails include those described in U.S. Pat. No. 4,622,959 to Marcus; U.S. Pat. No. 4,913,137 to Azer et al.; U.S. Pat. No. 5,281,224 to Faccioli et al.; U.S. Pat. No. 6,039,739 to Simon; U.S. Pat. No. 7,060,070 to Anastopoulos et al.; U.S. Pat. No. 7,077,847 to Pusnik et al.; U.S. Pat. No. 7,147,642 to Robioneck et al.; U.S. Pat. No. 7,311,710 to Zander; U.S. Pat. No. 7,232,443 to Zander et al.; and U.S. Pat. No. 7,549,994 to Zander et al. These devices typically include rigid arms that extend from the intramedullary nail that guide a drill bit toward an opening in the intramedullary nail. However, these devices fail to provide the degree of accuracy required for locating and drilling openings in intramedullary nails due to the inherent flexure in these devices. Furthermore, the flexure increases as the length of the arm increases, which renders them impractical for drilling distal openings in the nails. These devices can be deflected as much as a centimeter or more off the distal openings of an intramedullary nail.

The earliest successful magnetic targeting was accomplished by Durham et al. and was described in a succession of patents covering a mechanical magnetic targeting system using a mechanically balanced cannulated magnet (U.S. Pat. Nos. 5,049,151; 5,514,145; 5,703,375; and 6,162,228). Hollstien et al. (U.S. Pat. No. 5,411,503) followed with an electrically based system of stacked flux finders connected to a PC display. These devices, however, operate at the level of the skin. The magnets used in these devices may not be strong enough to accurately position the drill bit as even the fields of the strongest magnets diminish to that of the earth's magnetic field at distance of about 10 cm.

As a result, all of the prior devices have yet to be practical in surgical use.

### SUMMARY OF THE INVENTION

The present invention provides an intramedullary nail targeting apparatus.

A preferred version of the targeting apparatus includes a nail extension. The nail extension is capable of being connected to an end of an intramedullary nail and includes a targeting arm configured to extend along a longitudinal axis of the intramedullary nail when connected thereto. The targeting arm on the nail extension includes one or more bores.

The targeting apparatus also includes a magnetic targeting device capable of detecting a magnet for attaching to the targeting arm. The targeting arm provides support and stability for the magnetic targeting device. The magnetic targeting device includes a support member having a proximal end and a distal end and is structured to fit through at least one of the bores in the targeting arm, a sensor array disposed on the distal end of the support member, and a positional indicator. The support member has a length sufficient to place the sensor array against a bone comprising the intramedullary nail when the nail extension is connected to the intramedullary nail and the magnetic targeting device is connected to the targeting arm.

The targeting apparatus also includes a magnet member disposed in fixed relation to the intramedullary nail. Targeting of the magnetic targeting device to the magnet member in the intramedullary nail aligns the targeting arm on which the magnetic targeting device is supported with the intramedullary nail. This, in turn, aligns bores in the targeting arm with screw openings in the intramedullary nail. The bores can then be used for accurate drilling of the bone to secure the intramedullary nail thereto.

A preferred version of the invention further includes a magnet member that produces a radial magnetic field. This includes, for example, a magnet member comprising individual magnets in a “bucking configuration,” wherein the magnet member includes a first magnet and a second magnet arranged coaxially with like poles placed head-to-head. In other versions of the invention, the magnet member includes a third magnet interposed between the first and the second magnets and oriented orthogonally to the first and the second magnets.

Some versions of the invention further include an orthogonal targeting guide for targeting and drilling orthogonal screw openings in intramedullary nails. A preferred version of the orthogonal targeting guide includes a lateral support base for attaching to the targeting arm or other support structures, orthogonal support arms extending from the lateral support base, and a mechanical targeting arm with orthogonal guide bores for use in drilling the orthogonal screw openings. The orthogonal targeting guide also preferably includes a straight-edge guide for aligning the orthogonal guide bores over the orthogonal screw openings.

The invention also provides a method of targeting screw openings in an intramedullary nail for the internal fixation of a bone within a limb, wherein the intramedullary nail includes first and second screw openings. In a preferred version, the method includes placing the intramedullary nail in a medullary cavity of the bone, wherein the intramedullary nail includes a magnet member positioned at a known, fixed position relative to the second screw opening, attaching a nail extension comprising at least a first bore and a second bore to a proximal end of the intramedullary nail, attaching a magnetic targeting device to the targeting arm, aligning the magnetic targeting device with the magnet member, drilling a first hole in the bone at a position of the first screw opening, stabilizing the targeting arm to the first screw opening, and drilling a second hole in the bone at a position of the second screw opening. In this version, the second screw opening is targeted with the magnetic targeting device inserted through the second bore while the first screw opening is drilled using the first bore.

In some versions, the targeting arm is stabilized to the first and second screw openings after drilling the second hole. The targeting arm is preferably stabilized to the screw openings with drill guides. The stabilizing is followed by attaching an orthogonal targeting guide to the stabilized targeting arm and drilling holes in the bone through the orthogonal targeting guide.

Other versions further include un-stabilizing the targeting arm after drilling the second hole, rotating the nail extension orthogonally, targeting orthogonal openings in the intramedullary nail with the magnetic targeting device, and drilling holes in the bone through the orthogonal openings.

The invention further provides a bone plate targeting apparatus for targeting a bone plate including holes. The apparatus comprises a magnet member disposed a defined distance from at least one of the holes in the bone plate, and a magnetic targeting device.

The invention further provides a method of targeting holes in a bone plate for the external fixation of a bone within a limb. The method comprises placing the bone plate against the bone, placing a magnetic targeting device against the bone plate, aligning the magnet member with a sensor array in the magnetic targeting device, wherein aligning the magnet member with the sensor array aligns the lower opening of the drill guide with the at least one of the holes, and drilling a hole in the bone through the hole in the bone plate.

The present invention advantageously provides magnet members that provide larger magnetic fields in the same space confines, a targeting system that is unaffected by incidental rotation of a magnet member within an intramedullary nail, the ability to use smaller sensor arrays that can be used percutaneously while still attaining accurate targeting, a targeting system that can be used for a variety of bone sizes and intramedullary nail sizes, and a stable system that minimizes erroneous degrees of freedom while targeting and drilling.

The objects and advantages of the invention will appear more fully from the following detailed description of the preferred embodiments of the invention made in conjunction with the accompanying drawings.

### BRIEF DESCRIPTION OF THE DRAWINGS

FIG. 1 is a perspective view of the magnetic targeting device of the present invention.

FIG. 2 is a cross-sectional view of the magnetic targeting device of FIG. 1 taken along lines 2-2 of FIG. 1.

FIG. 3 is a cross-sectional view of the sensor foot of the magnetic targeting device of FIG. 1 taken along lines 3-3 of FIG. 2.

FIGS. 4A and 4B are partial side plan views of the magnetic targeting device of FIG. 1 comprising a hinged sensor foot.

FIG. 5 is a side plan view of the magnetic targeting device illustrating its operation with respect to a long bone.

FIG. 6 is a top view of the intramedullary nail of the present invention.

FIG. 7 is a top plan view of the magnetic targeting device of FIG. 1 with the cover (i.e., upper body portion) removed.

FIG. 8 is a block diagram illustrating the operation of the magnetic targeting device of the present invention.

FIG. 9 is a top plan view of the magnetic targeting device of FIG. 1 illustrating the display.

FIG. 10 is a diagram illustrating the amplitude output of the sensors.

FIG. 11 is a diagram illustrating the flux density of the magnetic field at various distances from the magnet.

FIG. 12A is a side cutaway view of a magnet member on a magnet insertion rod in a “bucking” configuration within an intramedullary nail.

FIG. 12B is a cross-sectional view taken across line 12B-12B of FIG. 12A.

FIG. 12C is a side cutaway view of a magnet member comprising both longitudinally and orthogonally oriented magnets on a magnet insertion rod.

FIG. 13 is a perspective view of a magnetic targeting device mounted on a nail extension of the present invention.

FIG. 14 is a perspective view of a magnetic targeting device mounted on a nail extension with an orthogonal targeting guide mounted on the nail extension.

### DETAILED DESCRIPTION OF THE INVENTION

### Definitions

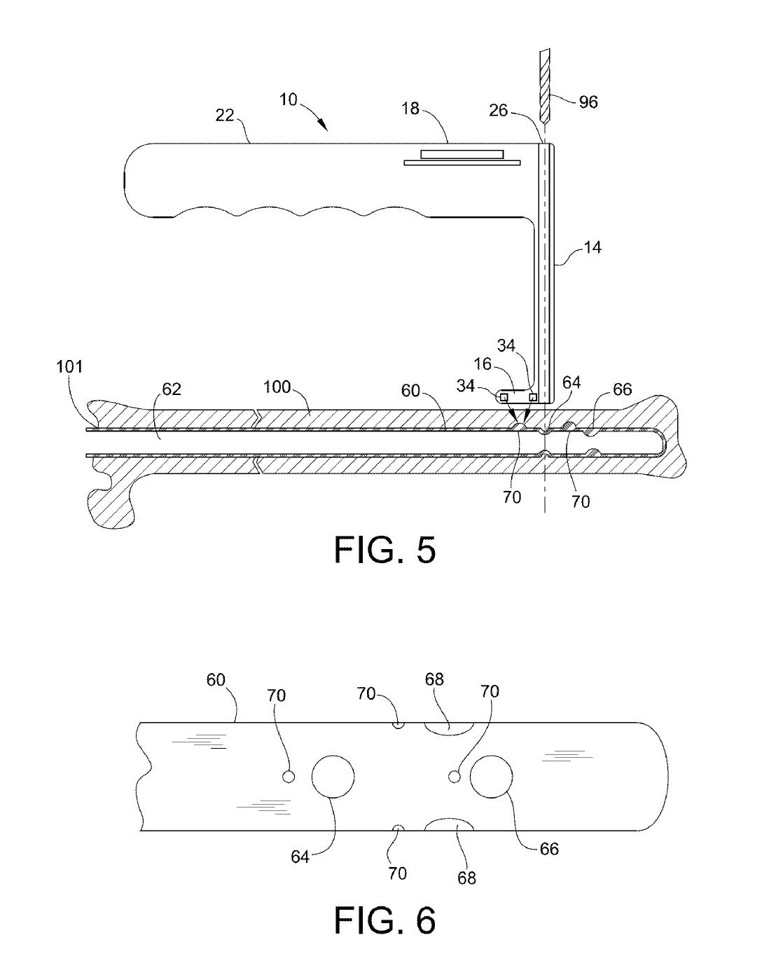

Unless explicitly stated otherwise, “x axis,” “y axis,” and “z axis” used in reference to the intramedullary nail 60 or the magnet member 70 inserted in the intramedullary nail 60 are defined relative to the intramedullary nail 60 having screw openings 64,66,68 shown in FIGS. 5 and 6. “X axis” refers to an axis defined by the long axis of the intramedullary nail 60. “Y axis” refers to an axis defined by the central axis of screw opening 68, which is substantially orthogonal to the long axis of the intramedullary nail 60 and to screw openings 64,66. “Z axis” refers to an axis defined by the central axis of screw openings 64,66, which are substantially orthogonal to the long axis of the intramedullary nail 60 and to screw opening 68. Thus, in FIGS. 5 and 6, the x axis runs the length of the depicted intramedullary nail 60 from its left-hand side to its right-hand side; the y axis runs perpendicular to the length of the depicted intramedullary nail 60 through screw opening 68; and the z axis runs perpendicular to the length of the depicted intramedullary nail 60 through screw openings 64,66.

### Magnetic Targeting Device 10

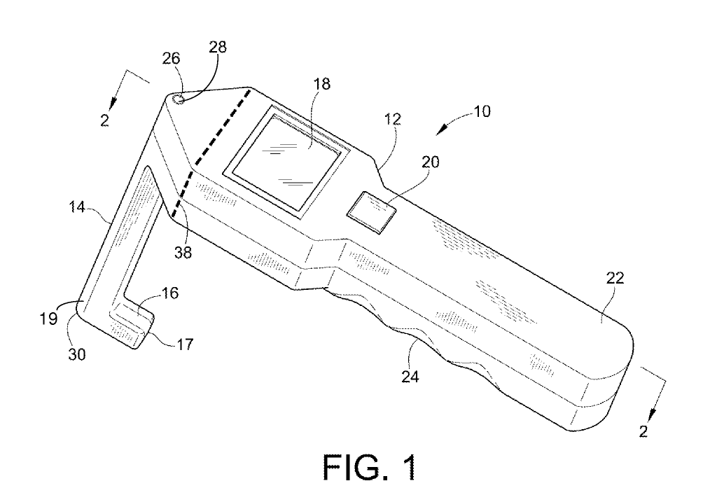

Referring now to FIG. 1, the present invention includes a magnetic targeting device 10 which, in an exemplary version, includes a body 12 with a handle portion 22, a support member 14, a button 20, a sensor foot 16 connected to a distal end of the support member 14, a display 18, and a drill sleeve 26 constituting or extending through the support member 14. The magnetic targeting device 10 places the sensor foot 16 of the support member 14 directly on the bone 100, illustrated in FIG. 5, for more accurate reading.

### Body 12

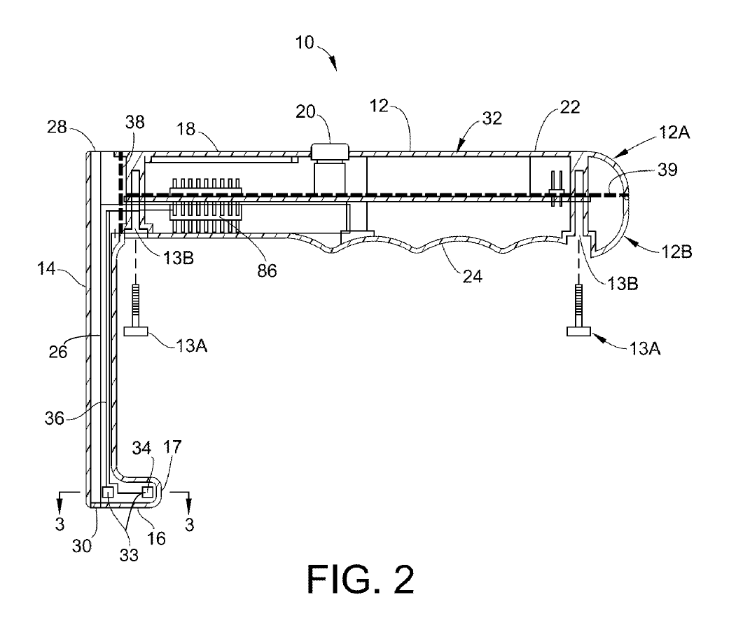

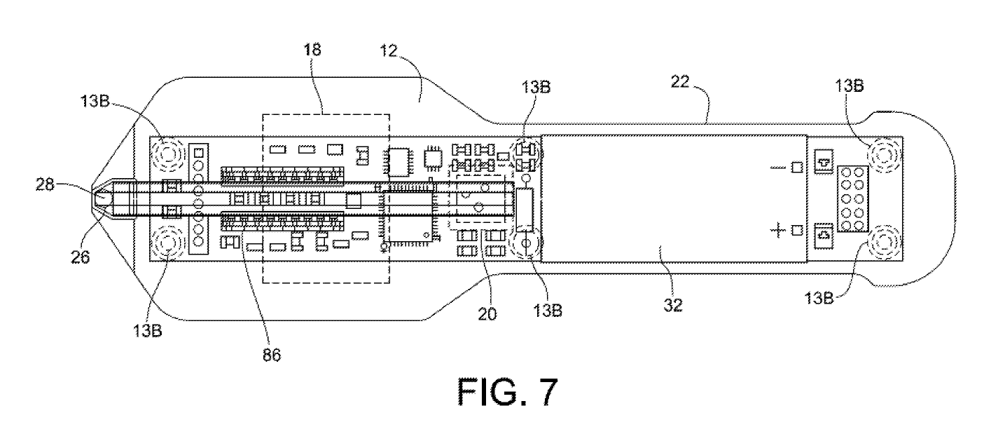

The body 12 can be made of a variety of materials known to the medical arts, including plastic and metal as appropriate for durability and reusability of the magnetic targeting device 10. As illustrated in FIG. 1, the body 12 is designed to be handheld and comfortable with finger grips 24 in the handle portion 22. The body 12 also holds the battery 32, the comparator circuit 86 and the display 18, as illustrated in FIGS. 2 and 7. The magnetic targeting device 10 can operate on two AAA batteries, have rechargeable cells, or be wired for electrical operation.

The body 12 of the magnetic targeting device 10 is amenable to several non-limiting design variations, each with various advantages.

In some versions, the body 12 and support member 14 are provided as a single unit.

In the exemplary version, the body 12 and support member 14 are provided as separate units and are separable, for example, at line 38 (see FIGS. 1 and 2). Connecting elements are known in the art for joining the support member 14 to the body 12 in a manner to enable the electrical connection between the two units. In the exemplary version, the body 12, which contains the electronic circuitry (such as the comparator circuit 86), may be provided in a sterile bag (not illustrated) and would not have to be sterilized prior to use. During use, the plastic bag containing the body 12 could be perforated by the sensor-support member 14 portion of the device to connect to the electronic circuitry in the body 12 to render the magnetic targeting device 10 ready for use. Alternatively, the electronics can be made to withstand sterilization, including but not limited to gas sterilization, autoclaving, CIDEX® disinfecting solutions (Johnson & Johnson Corporation, New Brunswick, N.J.) or other similar chemical soaks, or any equivalent thereof. This permits the support member 14 to attach to the body 12 at line 38 and be used without a sterile bag.

Having the support member 14 and the body 12 as separate units also allows for different interchangeable support member 14 options for the same body 12. One advantage of having different support member 14 options is that they can be used for different applications such as humeral or tibial nail-locking, which might use smaller diameter locking screws and require narrower drill sleeves 26. A second advantage is that support members 14 having different lengths may be used. Shorter support members 14 would allow more efficient use of the magnetic targeting device 10 when deep soft tissues do not have to be avoided. A third advantage is that different sensor array 33 configurations (see below) may be used for different applications. The ability to use different support member 14 options therefore prevents the necessity of making a different magnetic targeting device 10 for each application.

Providing the body 12 and support member 14 as separable units also permits the support member 14 to be made of disposable materials for simple disposal after use.

In another version, the magnetic targeting device 10 is connected wirelessly between the sensor foot 16 and the display 18 to transfer targeting or display information wherever needed. The sensing information may be transmitted by radio, infrared, or equivalent thereof from the sensor foot 16 to the display 18. The display 18 may be separate from the body 12 and can comprise any medium, including virtual projections, heads-up glasses, a personal computer, or a television screen. Such a display 18 can be made from any compatible non-magnetic material.

The body 12 may also be separable along line 39, as shown in FIG. 2, to divide the body 12 into an upper body portion 12A and a lower body portion 12B. The upper and lower body portions 12A,B, may be connected by screws 13A that insert into threaded holes 13B, the latter of which extend from the lower body portion 12B into the upper body portion 12A. Other mechanisms of connecting the upper and lower body portions 12A,B may be used. The ability to separate the upper and lower body portions 12A,B allows the user to access internal parts of the device 10, such as the battery 32 and the comparator circuit 86.

The body 12 may be provided with or without a handle portion 22.

The button 20 is provided generally on the top surface of the body 12 at a convenient location for the surgeon to power and calibrate the device 10. The button may also turn off the device 10. The button 20 is positioned for comfortable use. There may be a button 20 on either side of the handle portion 22 activating the same functions, to allow for left- or right-handed use.

### Support Member 14 and Sensor Foot 16

The preferred design of the present invention includes a support member 14 about 10 cm in length. While the length of the support member 14 is variable, a length of 10 cm incorporates most distal femoral soft tissue sleeves. For tibial and humeral applications, the support member 14 can be as short as 3-4 cm.

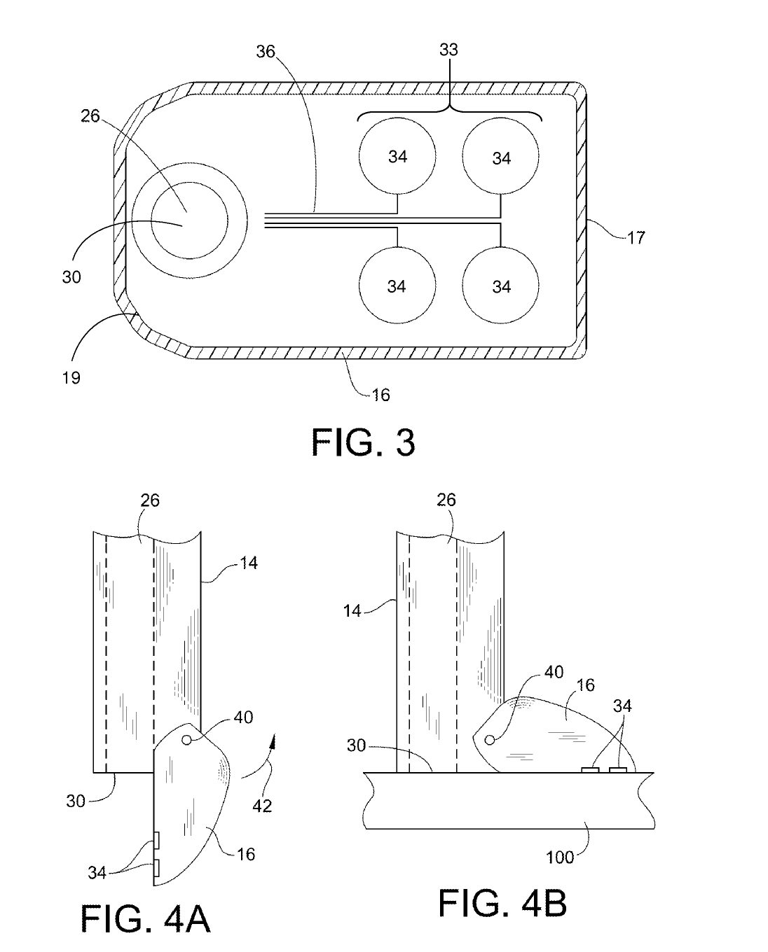

The sensor foot 16 is preferably disposed on a distal end of the support member 14 and comprises the sensor array 33. In a version shown in FIG. 3, the sensor foot 16 resembles a foot wherein the toe portion 17 contains the sensor array 33 and the heel portion 19 contains the lower opening 30 of the drill sleeve 26. In another version, the sensor foot 16 comprises the same shape as the distal end of the support member 14. A smaller sized sensor foot 16 on the support member 14 is more practical to use.

In some versions, the sensor foot 16 can be separated from the support member 14. This enables sensor feet 16 having different sensor arrays 33 to be used on the support member 14.

As shown in FIGS. 4A and 4B, some versions of the sensor foot 16 include a swivel design wherein the sensor foot 16 is hingedly attached to the support member 14 by means of a hinge unit 40. This configuration eases insertion of the sensor foot 16 into the soft tissues at the point of insertion. The hinge unit 40 can be made of a number of materials and designs to incorporate the swivel functioning of the unit. Prior to insertion into an opening in a limb for positioning next to a bone 100, the sensor foot 16 is rotated by means of the hinge 40 and pointed in parallel alignment with the support member 14 for ease of movement toward the bone 100, as illustrated in FIG. 4A. As the toe portion 17 comes in contact with the bone 100, the foot 16 will rotate in an arc approximating arrow 42 until the sensor foot 16 rests on the bone 100 approximately perpendicular to the support member 14, as illustrated in FIG. 4B.

### Sensor Array 33

The sensor array 33 is preferably included within the sensor foot 16 of the support member 14 near the lower opening 30 of the drill sleeve 26 (see FIG. 3). In one version of the invention, the sensor array 33 is dimensioned and configured such that each sensor 34 in the array 33 is capable of being excited by the same magnitude and angle of flux when centered about the magnet member 70. As used herein, “angle of flux” refers to the angle of the magnetic field 74 flux lines 78 relative to the orientation of the sensor 34 and does not refer to the direction through which the flux lines 78 run through the sensor 34. For example, sensors 34 positioned equidistantly from and on either side of a center line of flux 75 extending from a magnet member 70 would have the same magnitude and angle of flux even though the flux lines 78 would extend through the sensors 34 in opposite directions. An exemplary version of an array 33 that is excited by the same magnitude and angle of flux when centered about the magnet member 70 is shown in FIG. 3. The sensor array 33 in this version includes four magnetic sensors 34 arranged in a substantially planar, symmetrical array. Other exemplary substantially planar arrays include those described in U.S. Pub. No. 2005/0075562 to Szakelyhidi et al.

Other sensor arrays 33 may be symmetrical about the magnetic field 74 but not planar. For example, the sensor array 33 may include a pyramidal arrangement. Such an arrangement may include one or two additional, “z-axis” sensors positioned equidistantly from sensors 34 arranged in a planar, symmetrical arrangement. The z-axis sensors may be placed anywhere along an axis running through the center of the planar, symmetrical arrangement of sensors 34. In one version, the sensor array 33 includes one z-axis sensor positioned outside the plane defined by the sensors 34 arranged in the planar, symmetrical arrangement. In a second version, the sensor array 33 includes a first z-axis sensor positioned outside the plane defined by the sensors 34 in the planar arrangement and a second z-axis sensor positioned within the plane defined by the sensors 34 in the planar arrangement. The z-axis sensor positioned outside the plane in these versions is preferably disposed on a side of the planar sensors 34 opposite the magnet member 70. A sensor array 33 in a pyramidal arrangement provides both translational and rotational positional information with respect to the magnet member 70. When the sensor array 33 is aligned over the field, the z-axis sensors detect the field at maximum strength.

In sensor array 33 configurations comprising z-axis sensors, a magnet 72 placed at a distance from the sensor foot 16 may dispose the z-axis sensors between collinear flux lines 78. Targeting in such a case may be achieved when the sensors detect flux lines 78 parallel to the magnetic field 74.

The sensor array 33 may include any number of sensors 34 in any configuration, provided that each sensor 34 in the array 33, in combination with other elements of the invention, is capable of detecting the magnetic field 74 in a manner that predictably indicates the translational and/or rotational position of the magnetic targeting device 10 relative to the magnet member 70. For example, in preferred versions, the system permits translational alignment in either the x-y and/or x-z planes in addition to rotational alignment about the x, y, and z axes.

The individual sensors 34 in the sensor array 33 are preferably polarized sensors. As used herein, “polarized sensors” are sensors 34 capable of detecting the magnetic field 74 in all three dimensions (as defined by the sensor), thereby providing a readout of the magnitude and direction of the flux lines 78 comprising the magnetic field 74 at a given position. A preferred example of a polarized sensor that may be used in the sensor array 33 is a Honeywell HMC 1052 (Morristown, N.J.) magneto resistive sensor. Magneto resistive sensors advantageously have an internal magnetic reset function that can reverse the magnetizing effect of a permanent magnet when brought too close to the sensor array 33. This feature works well and is used to reset the sensors 34 upon every calibration operation (described below). The sensor reset driver pushes a large current pulse through all sensors at once to perform the reset.

The sensor array 33 is connected to the comparator circuit 86 in the body 12 by printed circuit wiring, wires 36 extending within the support member 14 beside the drill sleeve 26 (see FIG. 2), or through wireless communication. In the exemplary version shown in FIG. 2, the sensor array 33 is molded in a plastic support member 14 with the wires 36 from the sensor array 33 ascending the support member 14 to the comparator circuit 86 and linked to a display 18.

The magnetic targeting device 10 is preferably configured such that each individual sensor 34 in the sensor array 33 detects multiple flux lines 78 for high resolution in targeting. This is a difficult hurdle in conventional magnetic intramedullary nail targeting devices. All magnets obey the inverse square rule, wherein the strength of the magnetic field drops off at the square of the distance. Doubling the distance decreases the magnetic field strength to 25%. If the distance between a sensor and a magnet is 10 cm, the magnetic field is 1% the strength and field density of a sensor array 1 cm from the magnet. Conversely, the strength of the magnetic field at 1 cm from the magnet would be 100 times stronger than the same magnetic field measured at 10 cm.

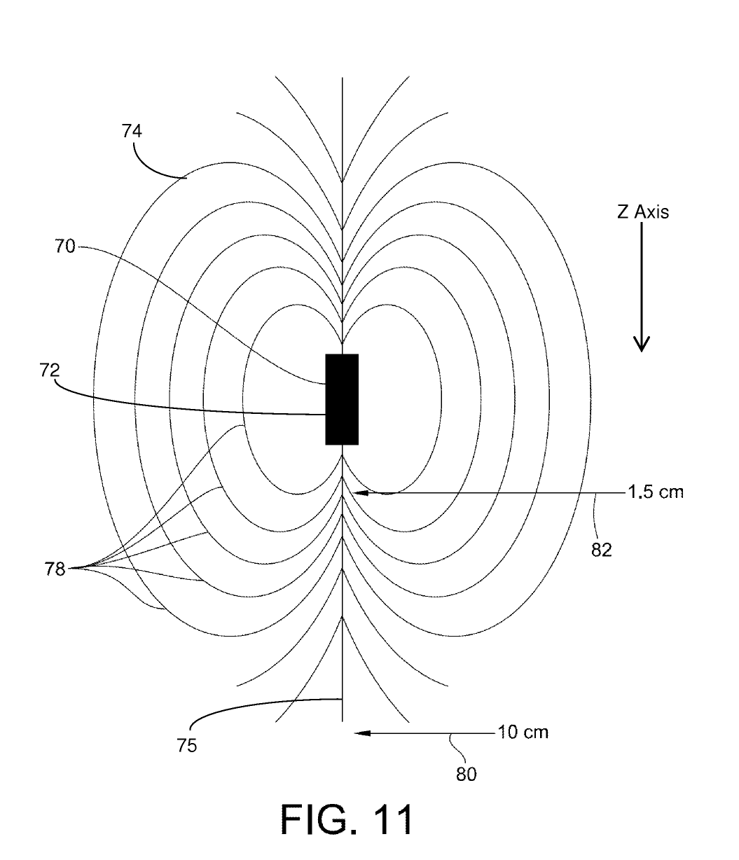

As shown in FIG. 11, the lines of flux 78 of a magnetic field 74 are so diffuse at a distance of 10 cm 80 from a magnet member 70 that a sensor would detect only one or fewer flux lines 78 at a time. This is insufficient for accurately locating the center of a 5 mm hole. At a distance of 1.5 cm 82 or other distances closer to the magnet member 70, multiple flux lines 78 can be detected and translated into targeting information. This applies even for relatively small sensors.

Disposing the sensor array 33 on the sensor foot 16 in the present invention allows the sensor array 33 to be placed at the surface of the bone 100 and in close proximity to the magnet member 70. As a non-limiting example, a sensor array 33 suitable for detecting multiple flux lines 78 in the current system includes individual sensors 34 1-2 mm square and arranged in an array 33 about 5-8 mm across and 2-5 mm thick. A preferred distance between the sensor array 33 and the magnet member 70 is a distance of about 1.5 cm, typically the average thickness of the side of the bone 100. At that distance, the field density is about 30 times the density at a distance of 10 cm. Other acceptable distances include about 1 cm, about 2 cm, about 3 cm, about 4 cm, about 5 cm, about 6 cm, or more. The center line of flux 75 of the magnetic field 74 can be offset as little as 6-10 mm from the center axis of the hole to be drilled. To date the most difficult distal targeting goal has been the distal femur. The working distances from the annular cavity 62 of an intramedullary nail 60 in a distal femur to the surface of the bone is typically no more than 3 cm and is usually 1-2 cm. Thus, the magnetic targeting device 10 described herein is capable of accurately targeting the distal femur. This makes targeting nearly any other bone, i.e., the tibia, humerus, or any other long bone, even easier with the magnetic targeting device 10 described herein because of smaller cortex to nail distances.

In a preferred version, the sensors 34 in the array 33 are positioned so that they are perpendicular to the maximum density flux lines when the array 33 is centered over the magnet member 70.

### Intramedullary Nail 60

Referring to FIG. 5, the magnetic targeting device 10 is illustrated in association with a long bone 100, such as a broken femur, tibia, or humerus bone. Within the bone 100, there is illustrated an intramedullary nail 60, known in the art. Examples of intramedullary nails are prevalent in the prior art. For example, reference is made to U.S. Pat. No. 6,503,249 to Krause and the patents to Durham (cited herein), the contents of which are incorporated herein for a description of intramedullary nail and manners of use. The intramedullary nail 60 is an elongated metal rod typically having an annular cavity 62; although, as described with respect to the intramedullary nail 60 in FIG. 6, the intramedullary nail 60 may also be a solid body. The intramedullary nail 60 typically includes a first, proximal screw opening 64 and a second, distal screw opening 66. The screw openings 64,66 of typical intramedullary nails 60 are transverse, i.e., having center axes about ninety degrees to the long axis of the nail 60, as illustrated in FIGS. 5 and 6. However, intramedullary nails 60 may contain non-transverse or oblique screw openings, i.e., having center axes at angles other than at about ninety degrees in relation to the long axis of the intramedullary nail 60. Intramedullary nails 60 also typically include one or more screw openings 68 positioned orthogonally to both the longitudinal axis of the nail 60 and screw openings 64,66, as illustrated in FIG. 6. As used herein, screw openings 64,66 are referred to as “lateral” screw openings 64,66, and screw opening 68 is referred to as an “orthogonal” screw opening 68.

Prior to placement of the intramedullary nail 60 within a bone 100, a reaming rod known to the art is worked through the medullary cavity 101 of the bone 100, such as a broken femur, tibia, or humerus bone. The intramedullary nail 60 is then placed within the medullary cavity 101 for securing within the bone 100 by means of cross-locking screws or bolts positioned through the screw openings 64,66,68.

### Magnet Member 70

The magnetic targeting device 10 of the present invention targets an intramedullary nail 60 by aligning the sensor array 33 on the magnetic targeting device 10 with a magnet member 70 in fixed relation to the intramedullary nail 60. The magnet member 70 comprises one or more individual magnets 72.

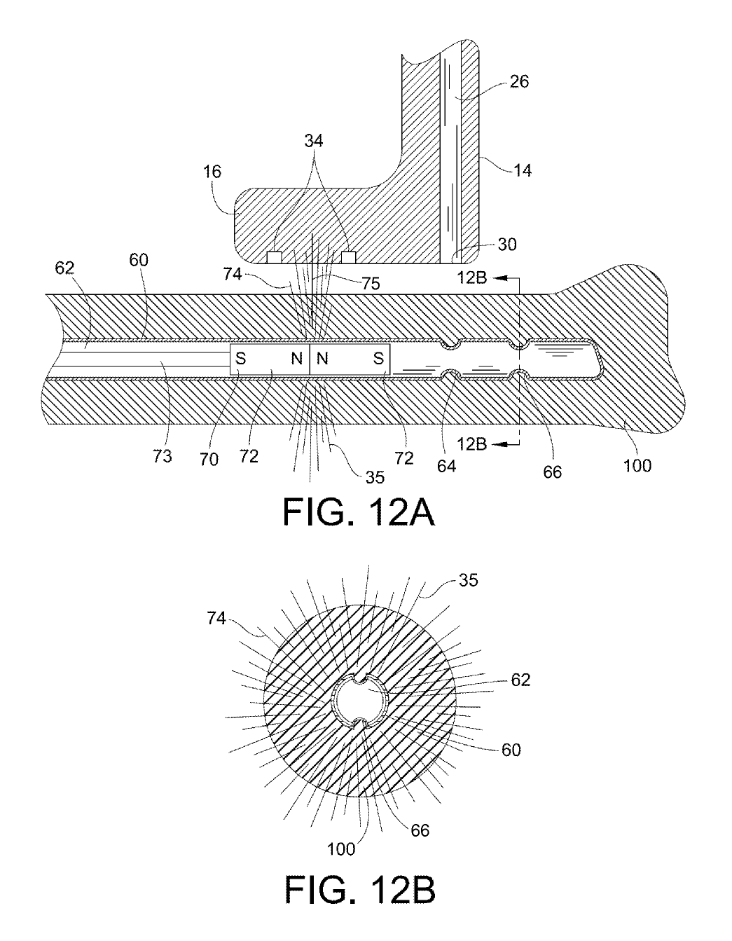

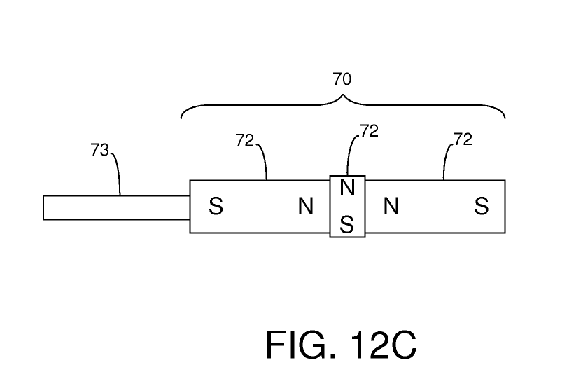

In a version of the invention shown in FIG. 12A, the magnet member 70 is attached to a magnet insertion rod 73 or other like device. The magnet insertion rod 73 is inserted into the annular cavity 62 of the intramedullary nail 60, typically in a specified orientation, to a locking point at a set distance from at least one of the screw openings 64,66,68. A reaming rod, known in the art, can be adapted for use as a magnet insertion rod 73. The adaptation requires a mechanism for attaching the magnet member 70 to the distal end of the rod 73, with provisions for maintaining correct depth, rotation, and centering of the magnet member 70 within the intramedullary nail 60. Such an attachment mechanism can include threads on a proximal end of the magnet insertion rod 73 that connect to a threaded portion of the annular cavity 60. The magnet insertion rod 73 can also be secured to an end of a nail extension 110 (see below). Magnet insertion rods 73 of different lengths can be included for placement of the magnet member 70 relative to different screw openings 64,66,68 along the length of the nail.

In another version of the invention, as illustrated in FIGS. 5 and 6, the intramedullary nail 60 has magnet members 70 embedded directly on the surface of the intramedullary nail 60. An intramedullary nail 60 with a magnet member 70 embedded therein does not require an annular cavity 62 and can be solid.

In another version (not shown), a magnetic ring is placed around the periphery of the screw openings 64,66,68 or to placed in the center of the screw opening 64,66,68 as a displaceable “bull's-eye.”

In yet another version (not shown), the magnet member 70 can be located at the screw opening 64,66,68 on a swivel that retracts when the drill enters the screw opening 64,66,68. The magnet member 70 is centered within the intramedullary nail 60 by a circular spring mechanism or equivalent.

In order to align and advance a drill bit 96 through the bone 100 accurately, a surgeon must have accurate knowledge of the position of the lower opening 30 of the drill sleeve 26 in relation to the axes of the screw openings 64,66,68. The magnetic targeting device 10 described herein accomplishes this by employing magnet member-sensor array 30-34 combinations that provide translational and/or rotational positioning information. For example, the magnet member-sensor arrays 30-34 described herein provide translational positioning alignment along planes orthogonal to the targeted screw openings 64,66,68, together with rotational positioning alignment about the central axis defined by the screw openings 64,66,68. Alternatively, the magnetic targeting device 10 employs magnet member-sensor array 30-33 combinations together with additional elements, such as a nail extension 110 (see below), to provide this alignment for targeting.

One version of the magnet member 70, shown in FIG. 11, employs a polarized magnet 72 with either its north or south pole facing an axis orthogonal to the x axis of the intramedullary nail 60 such that it projects a magnetic field 74 having a central line of flux 75 parallel to the axis of one of the screw openings 64,66,68. Such a magnet 70 may be dimensioned and configured to produce either circular or non-circular flux lines. Non-circular flux lines produce a non-circular field shape that uniquely defines each axis. This produces a field shape and polarity that potentially affords unique targeting information in all possible planes, such as the three-dimensional orientation of the intramedullary nail's 60 x-axis, y-axis, and z-axis. See U.S. Pub. No. 2005/0075562 to Szakelyhidi et al. regarding non-circular flux lines.

Another version of the magnet member 70, shown in FIGS. 12A and 12B, includes two individual magnets 72 with like poles placed head-to-head in a “bucking” arrangement. For example, a north pole of a first magnet 72 is connected to a north pole of a second magnet 72, and south poles of the first and second magnets 72 extend coaxially therefrom. The same arrangement can be achieved by placing the south poles head-to-head. The magnet member 70 in such an arrangement is preferably longitudinally oriented within the annular cavity 62 along the longitudinal axis (x axis) of the intramedullary nail 60. The bucking arrangement is advantageous in that it compresses the flux lines and produces a radial magnetic field 74 projecting orthogonally to the long axis of the intramedullary nail 60. Because the magnetic field 74 is radially projected, it always has a component perpendicular to the targeted screw openings 64,66,68, regardless of the amount of rotational deflection while inserting the magnet member 70 in the annular cavity 62 of the intramedullary nail 60. The condensed, radially projected magnetic field 74 also permits the sensor array 33 to be compressed, which, in turn, permits a smaller-sized sensor foot 16. This allows for placement of the sensor foot 16 directly against the bone 100 with less damage to surrounding tissue. Another advantage of the bucking arrangement is that the central lines of flux 75 emanating from the like poles of the magnet member 70 (FIGS. 12A and 12B) are at least twice the strength of central lines of flux 75 emanating from a magnet member 70 with its pole aligned orthogonally to the longitudinal axis of the intramedullary nail 60 (FIG. 11). This increases the strength of the magnetic field 74 at any given position on the z axis of the intramedullary nail 60.

The magnets 72 used in the bucking arrangement have cross-sectional dimensions and shapes that enable them to fit within the annular cavity 62 of the intramedullary nail 60. Most intramedullary nails 60 have an annular cavity 62 about 3-4 mm in diameter. The magnet 70 used in the bucking arrangement therefore are preferably sized with about 3 mm in cross-sectional width (i.e., diameter of a cylindrical-shaped magnet) and preferably no more than about 4 mm in cross-sectional width. This provides an optimal strength while still fitting in the annular cavity 62 of the intramedullary nail 60. However, it is within the scope of the present invention to use any size of magnet 72, as long as the magnet 72 can fit within the annular cavity 62 of the intramedullary nail 60.

Other magnet configurations for producing radially oriented magnetic fields 74 that can be used in the present invention are provided by U.S. Pat. No. 5,028,902 to Leupold et al. and U.S. Pat. No. 5,865,970 to Stelter.

Another version of the magnet member 70 is shown in FIG. 12C. This version comprises at least three magnets 72 disposed along a longitudinal axis, for example, the x axis of the intramedullary nail 60. Two of the magnets 72, comprising the ends of the magnet member 70, are disposed with both the north and south poles aligned along the longitudinal axis of the magnet member 70. These longitudinally oriented magnets are oriented with their like poles (i.e., north-north or south-south) facing each other, similar to the arrangement in the bucking configuration. A third, orthogonally oriented magnet 72 is interposed between the longitudinally oriented end magnets with its axis and central line of flux 75, parallel to the axis of one of the screw openings 64,66,68. In the preferred version of this magnet member 70, the longitudinally oriented magnets contact the orthogonally oriented magnet. However, the magnets may be separated by a short distance as well. As with the other magnet member 70 configurations, the magnet member 70 configuration shown in FIG. 12C can be attached co-axially along the longitudinal axis to a magnet insertion rod 73 for insertion in an annular cavity 62 of an intramedullary nail 60. The magnets 72 are each sized to fit within the annular cavity 62.

The magnet member 70 in the configuration shown in FIG. 12C produces a magnetic field 74 substantially similar in shape to a magnet member 70 comprising an orthogonally oriented magnet 72 alone (see FIG. 11). However, the presence of the longitudinally oriented end magnets tightens and further projects the magnetic field 74 along the axis defined by the orthogonally oriented magnet 72. The orthogonally oriented magnet 72 captures and redirects the “bucking” field preferentially toward the sensor array 33. The magnetic field produced by this configuration permits greater resolution in targeting at distances further away from the magnet member 72.

Several mechanisms can be employed to increase the sensitivity of the magnetic targeting device 10 with respect to the magnetic field 74. One mechanism includes superimposing a fluctuating magnetic field upon the static magnetic field 74 produced by the magnet member 70. Another mechanism includes placing a ferromagnetic material within the support member 14 between the sensor array 33 and the proximal end of the support member 14 on an axis running through the center of the sensor array 33. When in the presence of the magnetic field 74, the flux lines 78 concentrate on the ferromagnetic material, which extends the magnetic field 74 in the direction of the device 10.

Any type of magnet 72 may be used in the current device 10, including permanent magnets, solenoids, and electromagnets (i.e., iron core solenoids). A preferred version of the magnetic targeting device 10 includes a neodymium iron boron (NdFeB) bar magnet.

### Display 18

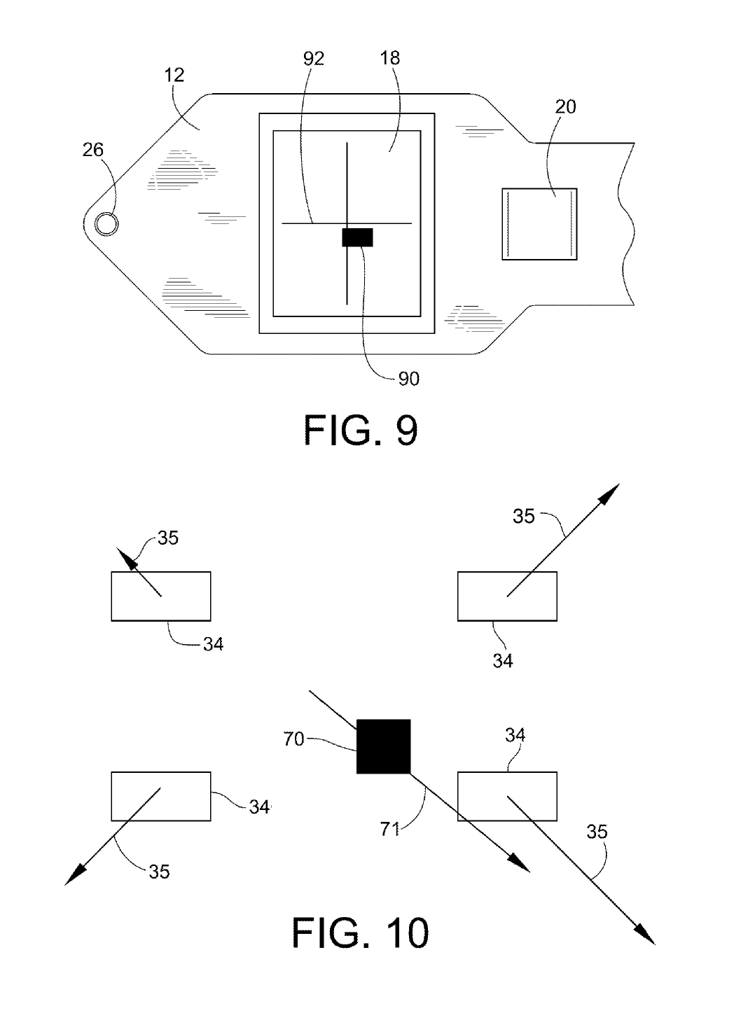

As illustrated in FIG. 9, the display 18 is preferably graphical in nature and provides a crosshair 92 in combination with a target icon 90. The crosshair 92 and target icon 90 indicate the amount of misalignment of the sensor array 33 with respect to the magnet member 70 in or on the intramedullary nail 60. Referring to FIG. 9, when the target icon 90 is centered on the crosshair 92, the sensor array 33 is centered over the magnet member 70. Depending on the version of the invention, this may indicate that the lower opening 30 of the drill sleeve 26 is centered over a screw opening 64,66,68 for accurate drilling. An advantage of this type of display is that it has sub-millimeter resolution. In addition, visualization of the position of the sensor array 33 relative to the magnet member 70 in the display 18 permits the surgeon to ultimately decide when drilling is appropriate. It is preferred that the display 18 includes a liquid crystal display (LCD) screen.

In addition to moving the target icon 90 with respect to the crosshairs 92, more accurate information can be attained by enlarging the target icon 90 in response to the strength of the magnetic field 74 being sensed. Being able to detect the strength of the magnetic field 74 at various locations ensures that the magnetic targeting device 10 is not sensing a symmetrical set of magnetic field 74 flux lines 78 around the magnet member 70 or a flux pattern created between two or more magnet members 70 which may be embedded into the side of a solid intramedullary nail 60.

Some versions of the magnetic targeting device 10 may include other types of positional indicators in addition to or as an alternative to the display 18 with crosshairs 92 and a target icon 90. These positional indicators may indicate positional information of the magnetic targeting device 10 relative to the intramedullary nail 60 and/or the magnet member 70 via any modality, including variable LED, audio output, color change, or vibration. In a version employing audio output, the magnetic targeting device 10 provides intermittent sounds such as beeps when the magnetic targeting device 10 detects a magnet field, with intervals between the intermittent sounds becoming shorter as the magnetic targeting device 10 becomes centered over the magnet member 70. In version employing a vibration modality, the magnetic targeting device 10 vibrates as the magnetic targeting device 10 first detects a magnetic field 74. The vibration grows in intensity as the magnetic targeting device 10 centers over the magnet member 70. Any of the display modalities described herein may be combined in any combination. For example, a magnetic targeting device 10 employing a visual display 18 may beep and/or provide a short vibration pulse upon the target icon 90 being centered on the crosshairs 92.

In other versions, the display 18 can operate in the manner described in U.S. Pub. No. 2005/0075562 to Szakelyhidi et al., which is incorporated herein by reference.

Some versions of the invention are capable of detecting positional information of the magnetic targeting device 10 relative to the intramedullary nail 60 and/or the magnet member 70 in three-dimensions, i.e., by detecting the position of the magnetic targeting device 10 relative to the x, y, and z axes of the intramedullary nail 60 and/or the magnet member 70. Such versions may provide positional indicators that reflect the three-dimensional position and orientation of the sensor array 33 relative to the magnet member 70. In one version, the positional indicator reflects the position of the magnetic targeting device 10 using two outputs. A first output displays the position with respect to a plane orthogonal to the targeted screw opening 64,66,68 (e.g., the x-y plane), and a second output displays the position with respect to a central axis defined by the screw opening 64,66,68 (e.g., the z axis). An example of a first output for such a positional indicator is as shown in FIG. 9. The translational positioning of the magnetic targeting device 10 on the x-y plane relative to the magnet member 70 is indicated by the positioning of the target icon 90 relative to the crosshairs 92. The rotational positioning of the magnetic targeting device 10 on the x-y plane relative to the magnet member 70 is indicated by rotation of the sides of the target icon 90 relative to the crosshairs 92. An example of a second output for such a positional indicator includes a line with a hash mark indicating the center of the line and a target icon positioned along the length of the line. Positioning of the rotational target icon along the line either to one side or the other of the hash mark would indicate rotational misalignment of the magnetic targeting device 10 relative to the z axis of the magnet member 70. Positioning of the rotational target icon on the hash mark would indicate alignment. The positional information afforded by such a positional indicator permits translational and/or rotational positioning with respect to the x-y plane and rotational position with respect to the z axis. This prevents off-axis drilling of the nail.

### Internal Operation of Device 10

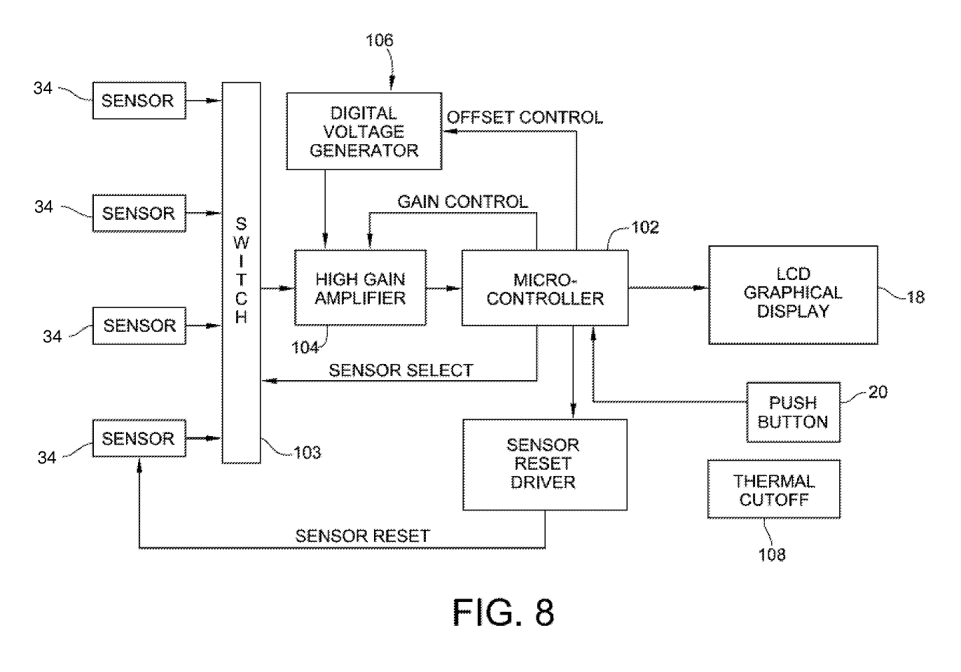

Reference is now made to FIGS. 7 and 8 for a description of the internal operation of the device 10. In action, the microcontroller powers a single sensor 34 in turn, using the switch 103 to connect it to the high gain amplifier 104. The microcontroller 102 then sets the digital voltage generator 106 to a predetermined value. The microcontroller 102 waits for the sensor 34 and amplifier 104 to settle and then reads the voltage from the amplifier 104. This voltage is proportional to the applied magnetic field 74 but also contains some environmentally generated noise and noise which is inherent in the sensors 34. The microcontroller 102 selects the four sensors 34 in sequence, measuring their outputs and saving them for targeting computations. A complete set of measurements is made typically 20 to 50 times per second. As with any high gain sensor system, small errors can be multiplied by factors of 1000 or more, resulting in problems making the required measurements. The sensors 34 are no different and have offset errors in their outputs that make measurements difficult without some adjustment. The amplifier 104 introduces errors as well. The digital voltage generator 106 is used during the calibration process to null out these errors.

When the magnetic targeting device 10 is powered on by the button 20, the magnetic targeting device 10 immediately begins a calibration sequence. This involves selecting each sensor 34 in turn and determining the value from the digital voltage generator 106 that is required to bring the amplifier 104 into its linear amplifying region of operation. This operation takes only a couple seconds. Thereafter, as each sensor 34 is selected, the digital voltage generator 106 is loaded with the particular value for that sensor 34, resulting in nullification of static errors for that sensor's measurement. The circuit also features a two-step amplifier gain selection, though the software may use only the high gain setting. Such a system allows use of the magnetic targeting device 10 for various thicknesses of human bone 100 without software changes. This design uses one amplifier 104 and an inexpensive commodity solid state switch 103 to select which sensor 34 to read. Another feature not shown is that the microcontroller 102 does not leave all sensors 34 powered continuously, but rather turns them on in sequence, saving power consumption.

The microcontroller 102 uses a vector algorithm to determine how to position the target icon 90 on the display 18. The position of each sensor 34 is assigned a vector direction depending on its position in the array 33. The amplitude of the output of each sensor 34 provides the magnitude of each vector 35. Addition of the magnitudes of the vectors 35 provide a resultant vector 71 that determines the position of the magnetic targeting device 10 relative to the magnet member 70, which is represented as a two-dimensional position of a target icon 90 on the display 18 (see FIG. 9). FIG. 10, for example, shows a center box representing the magnet member 70 and four other boxes representing the magnetic sensors 34. The vector lines 35 attached to each sensor 34, respectively, indicate the strength of the field at each sensor. The resultant vector 71 is the sum of the vector lines 35 and indicates the direction the sensor array 33 should be moved to center it over the magnet member 70. The magnet member 70 in FIG. 10 corresponds with the target icon 90 in FIG. 9.

The circuitry in the present invention compares and displays information about the magnetic field 74 in real time for rapid and accurate positioning of the targeting arm 120 while drilling.

Referring back to FIG. 8, the thermal cutoff 108 is present in case the magnetic targeting device 10 is accidentally run through a sterilizer cycle. The thermal cutoff 108 activates at 82° Celsius. and disables operation of the magnetic targeting device 10 permanently. Without the thermal cutoff 108, it is likely that the magnetic targeting device 10 would work somewhat after being exposed to such heat, but reliable operation could not be guaranteed. A low battery indicator is implemented that warns the user of low batteries 32 on the display 18 and also prevents the magnetic targeting device 10 from operating.

### User Operation

The button 20 is used to turn on the magnetic targeting device 10, and the magnetic targeting device 10 immediately performs a calibration cycle. If the button 20 is pressed briefly thereafter, another calibration cycle is initiated. The display 18 indicates to the user that calibration is in progress. It is not possible to turn on the magnetic targeting device 10 without initiating a calibration cycle. To turn off the magnetic targeting device 10, the button 20 is held down for a couple seconds until the display 18 goes off. The magnetic targeting device 10 also powers off after two minutes to prevent the batteries 32 from draining.

To perform targeting, the magnetic targeting device 10 is held in the same orientation as it will be used. The magnetic targeting device 10 is raised 10-12 inches above the targeting magnet member 70 and the button 20 is pressed to start a calibration cycle. It is important that the magnetic targeting device 10 be oriented approximately as it will be used in order to properly null the magnetic field of the earth. Once the magnetic targeting device 10 completes its calibration operation, it is lowered to the work area and moved to achieve an on-target indication.

### Nail Extension 110

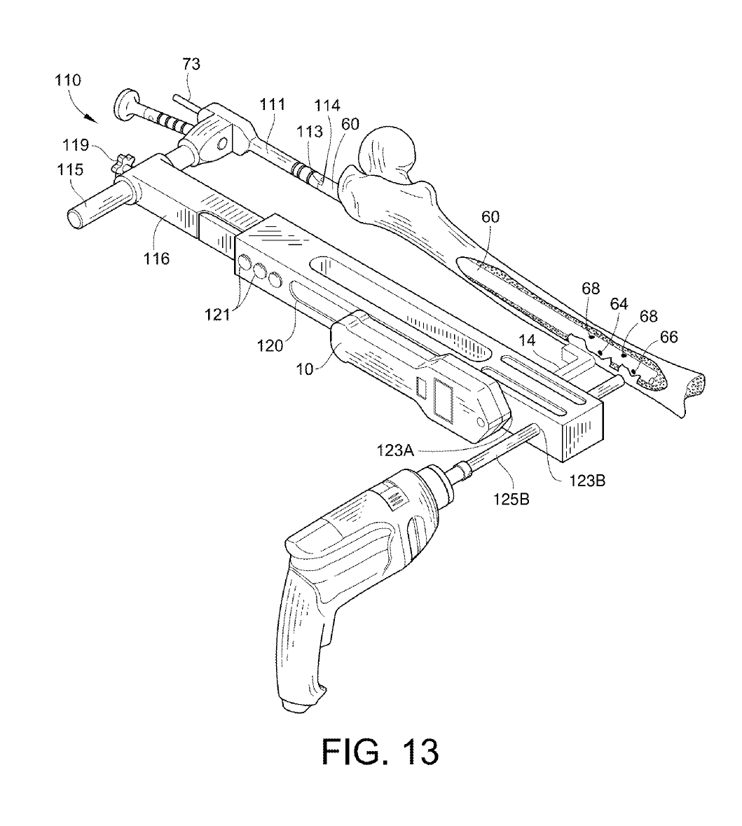

In a version of the invention as shown in FIG. 13, the magnetic targeting device 10 is included on a nail extension 110 of an intramedullary nail, the latter of which includes a nail connector 111 and a targeting arm 120. The nail extension 110 may be a continuous unit, or may be comprised of separate but attachable nail connector 111 and targeting arm 120 members.

The nail connector 111 is capable of being connected to a proximal end of an intramedullary nail 60 in a fixed rotational orientation around the x axis of the nail. The nail connector 111 may be connected to the nail by a threaded connection or in any other manner, all of which are well-known in the art. To maintain the fixed orientation, the nail connector 111 preferably includes diametrically aligned lugs 113 projecting from a surface of the nail connector 111 that interfaces with the intramedullary nail 60. The lugs 113 are shaped and sized to fit closely in respective recesses 114 in the proximal end of the intramedullary nail 60. Insertion of the lugs 113 within the recesses 114 during attachment of the nail connector 111 to the intramedullay nail 60 prevents rotation of the nail connector 111 with respect to the intramedullary nail 60 around the x axis.

The nail connector 111 further includes an annular cavity (not shown). When the nail connector 111 is connected to the intramedullary nail, the annular cavity of the nail connector 111 is co-axial and continuous with the annular cavity 62 of the nail. The annular cavity of the nail connector 111 and the annular cavity 62 of the nail are dimensioned and configured to accept a magnet insertion rod 73 therein. In a one version, a distal end of the annular cavity of the nail connector 111 and the annular cavity 62 at the proximal end of the nail are both threaded, and the magnet insertion rod 73 for insertion in these annular cavities 62 is externally threaded. The nail connector 111 is fastened to the nail 60 by threading the magnetic insertion rod 73 through both the annular cavity of the nail connector 111 and the annular cavity 62 of the nail 60. This threaded system permits the magnet member 70 on the end of the magnet insertion rod 73 to be placed at a known location at the distal end of the nail.

The nail connector 111 further includes a targeting-arm connector 116 that enables connection of the targeting arm 120 to the nail connector 111. In a preferred version, the targeting-arm connector 116 comprises a portion extending substantially parallel to the longitudinal axis of the nail. The distance between the nail 60 and the extended targeting arm 120 is preferably greater than the amount of tissue surrounding a patient's bone. This distance may be adjustable by a variety of mechanisms. In an exemplary version, the targeting-arm connector 116 is slidable along an orthogonally oriented portion 115 of the targeting arm 120 and secured thereto with a compression screw mechanism 119. The support member 14 preferably has a length sufficient to place the sensor array an appropriate distance from the magnet member 70 (see above) given the distance between the nail 60 and the extended targeting arm 120. The targeting-arm connector 116 preferably includes one or more connector holes for attaching the targeting arm 120 to the nail connector 111.

In one version of the invention, the nail connector 111 and targeting-arm connector 116 comprise the systems described in U.S. Pat. No. 7,232,433 and U.S. Pat. No. 7,549,994 to Zander et al., which are incorporated herein by reference.

The targeting arm 120 is preferably connected to the nail connector 111 via the targeting-arm connector 116 and extends substantially parallel to the longitudinal axis of the intramedullary nail 60. In the exemplary version, the targeting arm 120 may be fastened to the targeting-arm connector 116 with bolts 121 that insert through the targeting arm 120 and through the connector holes in the targeting-arm connector 116.

The targeting arm 120 includes a plurality of bores 123A,B. The targeting arm 120 preferably includes a corresponding bore 123A,B for each screw opening 64,66 in the nails 60 that are intended to be used with the targeting arm 120. The bores 123A,B are preferably coaxial with the corresponding screw openings when the targeting arm 120 is aligned with the intramedullary nail 60. One or more of the bores 123A,B may be dimensioned and configured to accommodate a support member 14, and one or more bores 123A,B may be dimensioned and configured to accommodate a drill sleeve 125. In the preferred version, the bores 123A,B are grouped in pairs comprising a proximal bore 123A and a distal bore 123B, wherein the proximal bore 123A accommodates a support member 14 and the distal bore 123B accommodates a drill sleeve.

The proximal bore 123A places the sensor foot directly over the magnet member 70 in the intramedullary nail 60 when the targeting arm 120 and the intramedullary nail 60 are aligned along the y and z axes. The fit of the support member 14 in the proximal bore 123A is snug enough to prevent lateral movement of the support member 14 in the proximal bore. This prevents misalignment of the targeting arm 120 relative to the intramedullary nail when the sensor foot 16 is aligned with the magnet member 70.

A proximal bore 123A with a magnetic targeting device 10 inserted therethrough may be used for magnetic targeting only or may also be used for drilling. When used for magnetic targeting and drilling, the proximal bore 123A is positioned on the targeting arm 120 such that alignment of the sensor foot 16 with respect to the magnet member 70 in the intramedullary nail 60 places the lower opening 30 of the drill sleeve 26 of the support member 14 directly over the corresponding screw opening, such as the proximal screw opening 64.

The distal bore 123B is configured to place a drill sleeve 125B directly over the corresponding screw opening, such as the distal screw opening 66, when the targeting arm 120 is aligned with the intramedullary nail 60. The fit of the drill sleeve 125B in the distal bore 123B is snug enough to prevent lateral movement of the drill sleeve 125B in the distal bore 123B. This permits accurate drilling through the distal bore 123B when the targeting arm 120 is aligned with the intramedullary nail 60.

In some versions of the invention, the targeting arm 120 has more than one proximal bore 123A and/or distal bore 123B. This permits targeting and drilling of each screw opening of intramedullary nails of difference sizes. A targeting arm 120 having more than one proximal bore 123A and/or distal bore 123B preferably has indicia along the length of the targeting arm 120 indicating the correct positions for targeting and drilling for a nail 60 of a particular size.

The support member 14 and the drill sleeve 125B preferably have substantially the same cross-sectional shapes and dimensions in the areas where each nests in the bores 123A,B. This permits all of the bores 123A,B in the targeting arm 120 to have the same dimensions and to accommodate either the support member 14 or the drill sleeve 125B therein. This allows different combinations of the bores 123A,B to be used for targeting and/or drilling. Alternatively, the support member 14 and the drill sleeve 125B are differently dimensioned and fit in bores 123A,B specifically designed to accommodate each.

It is preferable that the distal bore 123B is located on the targeting arm 120 far enough away from the proximal bore 123A so that the metal in the drill bit 96 while drilling through the distal bore 123B does not interfere with the magnetic field 74 generated by the magnet member 70. However, for purposes of drilling accuracy, it is important that the distal bore 123B is not placed too far from the proximal bore 123A. Because the intramedullary nail 60 and the targeting arm 120 are connected at their proximal ends, a small amount of misalignment at the position of a more proximal bore 123A results in a larger amount of misalignment at the position of a more distal bore 123B. Placing the proximal bore 123A just out of the range of interference induced by the drill bit 96 in the distal bore 123B minimizes such an amplification of misalignment.

The medullary cavity 101 of the femur is curved. Intramedullary nails 60 are therefore typically curved along their longitudinal axes for insertion in the medullary cavity 101. The targeting arm 120 may comprise a curvature that corresponds with the curvature of the intramedullay nail 60 such that each bore 123A,B in the targeting arm 120 is axially aligned with the screw openings in the nail 60 at approximately the same distance from the intramedullary nail.

During targeting and drilling, it is preferable to attach the magnetic targeting device 10 to the targeting arm 120 in some manner to prevent movement of the magnetic targeting device 10 with respect to the targeting arm 120. Such attachment is minimally achieved by virtue of inserting the support member 14 through the proximal bore 123A. Additional mechanisms of attachment may include snap-fit protrusions extending from the bottom of the nail connector 111 to fit into additional bores along the length of the targeting arm 120, zip ties, straps with “VELCRO”-brand hook-and-loop fasteners, and/or other fasteners. The targeting arm 120 may further include indented portions to nest the body of the device therein.

The nail extension 110 is preferably comprised of carbon fiber for maximum strength and minimum weight.

Y- and Z-Axis Alignment of Bores 123A,B in Nail Extension 110 with Radial Magnetic Field 74

The nail extension arm 110 does not admit of flexure along longitudinal axis of the targeting arm 120, i.e., “stretching.” Therefore, the targeting arm 120 is substantially fixed with respect to the x axis of the nail 60. However, the nail extension arm 110 does admit of flexure across the longitudinal axis of the targeting arm 120. In other words, the targeting arm 120 will yield slightly to forces having a z or y vector component. Because the targeting arm 120 is anchored via the nail connector 111 to the intramedullary nail 60, purely translational displacement of the sensor array 33 with respect to the magnet member 70 does not occur. Any flexure of the targeting arm 120 will therefore induce rotational misalignment with respect to the magnetic field 74. The rotational misalignment is read as an imbalance by the sensor array 33. This is true even when a symmetrical, planar array 33 of four sensors 34 and a magnet member 70 producing a radial magnetic field 74 is used. The detected imbalance can be corrected by positional adjustment of the targeting arm 120 relative to the intramedullary nail 60.

### Orthogonal Targeting Guide 130

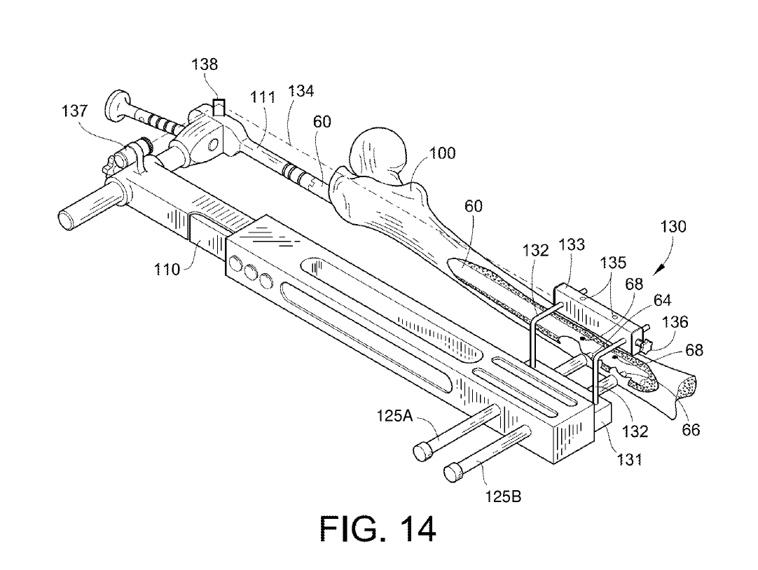

As shown in FIG. 14, some versions of the invention further include an orthogonal targeting guide 130, which is configured for use with the nail extension 110. The magnetic targeting device 10 is used to attach two parallel, mechanically stabilized drill sleeves 125A,125B against a lateral portion of the bone 100. The drill sleeves 125A,125B are stabilized at one end by the targeting arm 120 and at another end with set screws that fasten into holes drilled at the screw openings 64,66,68. Fastening the drill sleeves 125A,125B generates a stable, substantially rectangular construct comprising the stabilized drill sleeves 125A,125B, the targeting arm 120, the nail connector 111, and the intramedullary nail 60.

The orthogonal targeting guide 130 includes a lateral support base 131, orthogonal support arms 132, a mechanical targeting guide 133, and, optionally, a straight-edge guide 134. The lateral support base 131 attaches to the two parallel, mechanically stabilized drill sleeves 125A,125B, preferably by clamping thereto. The orthogonal support arms 132 extend from the lateral support base 131 to either the anterior or posterior side of the intramedullary nail 60 being targeted in a manner that clears soft tissues surrounding the bone 100. The orthogonal support arms 132 include the mechanical targeting guide 133 slidingly engaged thereto, such that the mechanical targeting guide 133 is capable of sliding on the orthogonal support arms 132 along the y axis of the intramedullary nail 60. The mechanical targeting guide 133 includes one or more orthogonal guide bores 135 that correspond to the position of the orthogonal screw openings 68 along the x axis, in addition to a locking screw 136 that restricts movement of the mechanical targeting guide 133 on the orthogonal support arms 132 along the y axis. The straight-edge guide 134 is mounted on the nail extension 110 and projects a physical or visual indicator of the midline of the intramedullary nail 60 for alignment of the orthogonal guide bores 135 on the mechanical targeting guide 133 with respect to the orthogonal screw openings 68 in the nail 60. In the exemplary version of the invention, the strait-edge guide 134 is a laser 137 that projects a visual indicator of the midline of the intramedullary nail 60. The laser 137 may be used with or without a mirror 138 also mounted on the nail extension 110. The orthogonal targeting guide 130 aligns the orthogonal guide bores 135 with the underlying orthogonal screw openings 68 in the intramedullary nail 60 for accurate drilling.

As an alternative to anterior-posterior targeting with an orthogonal targeting guide 130, the nail extension 110 may be configured to rotate to either an anterior or posterior position for targeting and drilling. In this version, the targeting arm 120 further includes bores positioned along the length of the targeting arm 120 to correspond to the position of the orthogonal screw openings 68 along the length of the intramedullary nail 60. Orthogonal recesses for accepting the lugs 113 are also included in the proximal portion of the nail 60 for maintaining the orientation of the targeting arm 120 in the xy plane.

### Intramedullary Nail 60 Targeting

In a preferred version of the invention, the proximal screw opening 64 is targeted while the distal screw opening 66 is drilled. This prevents magnetic interference from the drill bit 96 from disrupting targeting. The intramedullary nail 60 is placed in the marrow of the bone 100 and urged through the bone 100 as described in Szakelyhidi et al. The proximal opening 64 in the intramedullary nail 60 to be targeted has a magnet member 70 placed at a reproducible distance therefrom. The magnet member 70 is either embedded in the surface of the intramedullary nail 60 as illustrated in FIG. 6 or is inserted in the annular cavity 62 of the intramedullary nail 60 with a magnet insertion rod 73 and locked in place. A nail extension 110 with a nail connector 111 and a targeting arm 120 is attached to the intramedullary nail 60. The indicia on the targeting arm 120 indicate the end of the intramedullary nail 60, the approximate location of the openings 64, 66 in the intramedullary nail 60 in the bone 100, and the proximal bore 123A and the distal bore 123B in the targeting arm 120 that correspond with the proximal opening 64 and distal opening 66, respectively. An incision is made in the limb in the vicinity of the openings 64,66 according to the positions of the indicia. An oval trochar can be used to make a path for the support member 14 down to the surface of the bone 100. The support member 14 is inserted through the proximal bore 123A, and the sensor foot 16 is placed on the surface of the bone 100. In addition, a drill sleeve 125B is inserted through the distal bore 123B and placed directly on the bone 100. A drill bit 96 is then inserted into the drill sleeve 125B. A star-point drill prevents the drill from “walking” on the slippery curved surface of the bone and is therefore preferred.

While the distal bore 123B in the nail extension 110 places the drill sleeve 125B in the general vicinity of the distal opening 66, targeting at the magnet member 70 in the general vicinity of the proximal opening 66 corrects the final 2-3 mm misalignments resulting from the flexure of the nail extension 110. The sensor array 33 is activated to locate the magnet member 70, which then determines the location of the proximal opening 64. The display 18 is activated by the action of the button 20. A signal is sent to the sensor array 33 to zero the sensors 34. When the sensor array 33 is moved across the surface of the bone 100, the sensor information appears on the display 18, generally in the form of a target icon 90 and crosshairs 92 as illustrated in FIG. 9. If the sensor configuration affords z axis alignment information, a target icon 90 on a z-axis line in the display 18 also appears. The positioning of the target icon 90 in the center of the targeting grid 92 and positioning of the target icon 90 in the center of the z-axis line indicates correct placement of the magnetic targeting device 10 for drilling.

As soon as the target icons 90 align at the center of the crosshairs 92 and/or the z-axis line, the drill 96 is drilled through the distal opening 66 to the opposite cortex. The drill is far enough from the magnet member 70 and sensor foot that it does not produce magnetic interference.

Once the drill has passed through the bone cortex surrounding the distal opening 66, it is left in place. A modified drill sleeve 125B with a set screw is pushed against the cortex of the bone. The set screw is tightened, making a stable, substantially rectangular construct comprising the stabilized drill sleeve 125B, the targeting arm 120, the nail connector 111, and the intramedullary nail 60. With the distal opening 66 successfully targeted and stabilized, all proximal holes are aligned with the targeting arm 120. Drilling the proximal opening 64 occurs either by drilling through the drill sleeve 26 in the support member 14 of the magnetic targeting device 10 or by replacing the magnetic targeting device 10 in the proximal bore 123A with a separate drill sleeve 125A and drilling therethrough. Any other openings on the proximal side of the drilled and stabilized opening 66 are similarly drilled. The user has two options for targeting and drilling orthogonal openings 68, if drilling of such openings is desired. In a first option, the stabilized drill sleeve 125B at opening 66 is removed. The nail extension 110 is rotated 90 degrees about the x axis of the intramedullary nail 60. If using a magnet member 70 with its pole aligned orthogonally to the longitudinal axis of the nail 60, the magnet insertion rod 73 is also rotated 90 degrees about the x axis of the intramedullary nail 60. If using a magnet member 70 in a bucking arrangement, no rotation is required. If using a magnet member 70 embedded in the surface of the nail 60, the magnet member is pre-positioned for targeting and drilling. The orthogonal openings 68 are then targeted and drilled through orthogonal guide bores 135 corresponding with the orthogonal openings 68 in the same manner in which the lateral openings 64,66 were drilled.

In a second option, a second stabilized drill sleeve 125A is constructed at the proximal opening 64 such that there are two parallel, mechanically stabilized drill sleeves 125A,125B braced by the nail extension 110 and the intramedullary nail 60. An orthogonal targeting guide 130 is attached to the stabilized drill sleeves 125A,125B with the orthogonal support arms 132 directed to the desired side for drilling. A straight-edge guide 134, such as a laser 137, is mounted on the nail extension 110, and the anterior-posterior guide bores 135 are aligned with the straight-edge guide 134 to indicate the position of the underlying orthogonal openings 68 along the y axis of the nail 60. The orthogonal openings 68 are then drilled via mechanical targeting of the orthogonal targeting guide 130.

In some applications it is advantageous to insert a locking screw through the drilled opening 64,66,68 directly after targeting and drilling. A calibration on the drill measures the depth of the drilled hole at the upper opening 28 of the support member 14. Alternatively, after drill removal, the magnetic targeting device 10 can remain against the bone 100. A depth gauge is used to measure the length of the screw to be inserted. Once measured, the screw of the appropriate length is loaded onto a screw driver and inserted across the openings 64,66,68 of the intramedullary nail 60. Self tapping screws are used in the preferred embodiment.

An aiming device is always more accurate if it has two references in space to align it. In the present invention, a first reference to provide accuracy comes from the bores 123A,B on the targeting arm 120, which indicate the entry point on the skin directly over the opening 64,66,68 to be targeted in the intramedullary nail 60. The targeting arm 120 shows the correct entry point over each opening and stabilizes the device perpendicular to the longitudinal axis of the intramedullary nail 60. A second reference is provided by the magnetic targeting device 10, which is placed directly on the surface of the bone 100 to be targeted. The targeting of the magnetic targeting device 10 at the surface of the bone 100 corrects the final 2-3 mm misalignments resulting from the tolerances of the nail extension 110. The importance of being able to rest the magnetic targeting device 10 on the surface of the bone 100 during use cannot be over-emphasized. The accuracy needed for drilling and stabilizing intramedullary nails 60 within a broken bone is on the order of 1 mm. Use of either a magnetic targeting device 10 or mechanical targeting arm 120 alone is not as accurate as using both in combination.

### Bone Plates and Bone Plate Targeting

Versions of the device described herein can be extended to subcutaneous bone plating. Bone plates are generally solid, rigid plates with holes that attach to the outer surface of a bone, particularly a broken bone, to stabilize it. Bone plates are well known in the art. Examples include those described in U.S. Pat. No. 7,635,365 to Ellis et al. Bone plates used in the art are modified to include a magnet member 70 for targeting. In one version, a magnet member 70 is embedded in the surface of the plate proximal to a hole to be targeted for drilling the underlying bone 100. Preferably, the most distal drill hole of every plate has a 2 mm magnet member 70 embedded into the plate just proximal to the hole. In another version, a ring magnet is embedded around the hole. In either case, the magnet members 70 included in the bone plates are disposed on the outside of the bone 100. This enables the sensor foot to be placed in a percutaneous manner in the direct vicinity of the magnet member. Because the targeting distances are so small, a sensor foot 16 including a single sensor 34 can be used for targeting.

For targeting and drilling bone plating holes, the magnetic targeting device 10 is used either with or without an intramedullary nail 60 and nail extension 110. To target the bone plate with the device 10, a drill sleeve 26 is inserted in the support member 14, and the sensor foot 16 of the support member 14 is placed in the vicinity of the distal hole to be drilled. When the sensor foot 16 is aligned with the magnet member 70, the display is centered, and the distal hole is drilled. A modified Cleco spring fastener (Cleco Industrial Fasteners, Inc., Harvey Ill., USA) is inserted in the drilled hole to provide temporary fixation and stability. If the location of the drilled hole is correct after reduction of the fracture, the Cleco spring fastener is replaced by a screw. The Cleco spring fastener allows easy repositioning and drilling if minor adjustments in position of the plate are needed.

In an alternate version, drill holes in a subcutaneous bone plate are located by detecting threaded magnet members 70 that are screwed into holes pre-selected for use. The magnets 72 comprising the magnet members 70 are preferably NbFeBoron magnets for maximum strength. The magnet members 70 preferably have a hex drive. Because the most advantageous hole to locate during bone plating is the most distal subcutaneous hole of the plate, a magnet member 70 is inserted in the most distal hole. The magnets members 70 are sensed through the soft tissues by a sterile magnetic compass. Once located, the skin is marked and excised. The pre-positioned magnet members 70 in the screw holes are located by a magnetic screwdriver of the opposite polarity that locks into the hex head of the magnet member. Once the targeted hole is located, a hole is drilled, and a Cleco plate holder is inserted for immediate temporary fixation. If x-rays show that the reduction is satisfactory, other critical holes are located in a similar fashion. The distal Cleco plate holder is then removed and replaced by a locking screw. If the position of the plate is not ideal, the Cleco plate holder allows rapid repositioning of the distal end of the plate. The magnet-to-magnet location of the screw holes provides simplicity, low cost, and reliability in locating bone plating holes.

Plates made by Synthes, Inc. (West Chester, Pa., USA) have a combination of holes that are immediately adjacent to each other. In targeting such plates, one of the holes is modified to include a magnet member 70 and is used for targeting. A second hole is drilled through an adjacent parallel drill sleeve stabilized by the targeting arm 120. For single-hole plate designs, a magnet member placed in a small recess in the plate would allow a drill sleeve with a magnetic material to locate and lock into position for drilling.

Any version of any component or method step of the invention may be used with any other component or method step of the invention. The elements described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference in their entirety to the same extent as if each individual reference were specifically and individually indicated as being incorporated by reference. In case of conflict between the present disclosure and the incorporated references, the present disclosure controls.

The devices and methods of the present invention can comprise, consist of, or consist essentially of the essential elements and limitations described herein, as well as any additional or optional steps, ingredients, components, or limitations described herein or otherwise useful in the art.

It is understood that the invention is not confined to the particular construction and arrangement of parts herein illustrated and described, but embraces such modified forms thereof as come within the scope of the following claims.

I claim:

1. An intramedullary nail targeting apparatus including:
a nail extension capable of being connected to an end of an intramedullary nail and including a targeting arm configured to extend along a longitudinal axis of the intramedullary nail when connected thereto, the targeting arm including one or more bores;
a magnetic targeting device capable of detecting a magnet including:
a support member having a proximal end and a distal end and being structured to fit through at least one of the one or more bores in the targeting arm;
a sensor array disposed on the distal end of the support member; and
a positional indicator; and
a magnet member disposed in fixed relation to the intramedullary nail, wherein the support member has a length sufficient to place the sensor array against a bone comprising the intramedullary nail when the nail extension is connected to the intramedullary nail and the magnetic targeting device is connected to the targeting arm.

2. The apparatus of claim 1 wherein the magnet member produces a radial magnetic field.

3. The apparatus of claim 1 wherein the magnet member includes a first magnet and a second magnet arranged coaxially with like poles oriented head-to-head.

4. The apparatus of claim 1 wherein the magnet member includes a third magnet interposed between the first and the second magnets and disposed orthogonally to the first and second magnets.

5. The apparatus of claim 1 wherein the magnet member is coaxially disposed on the end of a magnet insertion rod, wherein the magnet insertion rod is dimensioned and configured to be fixedly inserted into an annular cavity of an intramedullary nail.

6. The apparatus of claim 1 wherein the magnet member is no more than 4 mm cross-sectional width.

7. The apparatus of claim 1 wherein the magnet member is embedded in the intramedullary nail.

8. The apparatus of claim 1 wherein the sensor array comprises sensors in a planar, symmetrical arrangement.

9. The apparatus of claim 8 wherein the sensor array further comprises a first additional sensor equidistant from each of the sensors in the planar, symmetrical arrangement

10. The apparatus of claim 9 wherein the first additional sensor is disposed outside a plane defined by the sensors in the planar, symmetrical arrangement.

11. The apparatus of claim 9 wherein the sensor array further comprises a second additional sensor equidistant from each of the sensors in the planar symmetrical arrangement.

12. The apparatus of claim 1 wherein the support member comprises a ferromagnetic material disposed within the support member between the sensor array and the proximal end of the support member on an axis running through a center of the sensor array.

13. The apparatus of claim 1 wherein the sensor array comprises polarized sensors capable of detecting and distinguishing x, y, and z vectors of a magnetic field.

14. The apparatus of claim 1 wherein the support member comprises cross-sectional width no more than about 9 mm.

15. The apparatus of claim 1 wherein the support member comprises a drill sleeve.

16. The apparatus of claim 1 wherein nail extension and/or the targeting arm is comprised of carbon fiber.

17. The apparatus of claim 1 wherein the targeting arm comprises a curvature

18. The apparatus of claim 1 further comprising an intramedullary nail that connects to the nail extension, wherein the targeting arm and intramedullary nail both comprise a curvature along their longitudinal axes and the curvature of the targeting arm corresponds to the curvature of the intramedullary nail such that the intramedullary nail is disposed a same distance from the targeting arm at each point along its longitudinal axis when the intramedullary nail is connected to the nail extension.

19. The apparatus of claim 1 further comprising an intramedullary nail that connects to the nail extension, wherein the intramedullary nail comprises a longitudinal axis and one or more screw openings along the longitudinal axis, wherein each screw opening in the one or more screw openings in the intramedullary nail has a corresponding bore in the one or more bores in the targeting arm.

20. The apparatus of claim 19 wherein each screw opening in the one or more screw openings in the intramedullary nail has a central axis coaxial with a central axis of the corresponding bore in the one or more bores in the targeting arm when the targeting arm is aligned with the intramedullary nail.

21. The apparatus of claim 1 further comprising a straight-edge guide mounted on the nail extension and defining an axis corresponding to a midline of an intramedullary nail.

22. The apparatus of claim 21, wherein the straight-edge guide is a laser.

23. The apparatus of claim 22 further comprising a minor mounted on the nail extension to direct the laser along the axis corresponding to the midline of the intramedullary nail.

24. The apparatus of claim 1 wherein the nail extension comprises an annular cavity for insertion of a magnetic insertion rod therethrough.

25. The apparatus of claim 1 wherein the positional indicator is a display disposed on the proximal end of the support member.

26. A method of targeting screw openings in an intramedullary nail for internal fixation of a bone within a limb, wherein the intramedullary nail includes first and second screw openings, the method comprising:
a. placing the intramedullary nail in a medullary cavity of the bone, wherein the intramedullary nail includes a magnet member positioned at a known, fixed position relative to the second screw opening:
b. attaching a nail extension to a proximal end of the intramedullary nail, wherein the nail extension includes a targeting arm extending a substantially consistent distance from a longitudinal axis of the intramedullary nail, the targeting arm including a first bore and a second bore, wherein the first bore includes a central axis that is configured to be substantially coaxial with a central axis of the first screw opening when the targeting arm is aligned with the intramedullary nail, and the second bore includes a central axis that is configured to be substantially coaxial with a central axis of the second screw opening when the targeting arm is aligned with the intramedullary nail; and
c. attaching a magnetic targeting device to the targeting arm, wherein the magnetic targeting device includes:
a support member having a proximal end and a distal end and being structured to fit through the second bore in the targeting arm;
a sensor array disposed on the distal end of the support member; and
a positional indicator,
wherein the support member is inserted through the second bore with the distal end of the support member positioned against the bone;
d. aligning the magnetic targeting device with the magnet member, wherein the aligning the magnetic targeting device with the magnet member aligns the targeting arm with the intramedullary nail;
f. drilling a first hole in the bone at a position of the first screw opening;
g. stabilizing the targeting arm to the first screw opening; and
h. drilling a second hole in the bone at a position of the second screw opening.

27. The method of claim 26 wherein the second screw opening is a proximal screw opening and the first screw opening is a distal screw opening, wherein the proximal screw opening and the distal screw opening are defined with respect to the proximal end of the intramedullary nail.

28. The method of claim 26 wherein the targeting arm is stabilized to the first screw opening with a first drill guide extending from the first screw opening through the first bore.

29. The method of claim 26 further comprising after step (h):
i. stabilizing the targeting arm to the second screw opening;
j. attaching an orthogonal targeting guide to the stabilized targeting arm; and
k. drilling holes in the bone through the orthogonal targeting guide.

30. The method of claim 29 wherein the targeting arm is stabilized to the second and first screw openings with a first drill guide extending from the first screw opening through the first bore and a second drill guide extending from the second screw opening through the second bore, wherein the orthogonal targeting guide is attached to the first and second drill guides.

31. The method of claim 29 further comprising between steps (j) and (k):
l. indicating a midline of the intramedullary nail with a straight-edge guide.

32. The method of claim 26 further comprising after step (h):
m. rotating the nail extension orthogonally;
n. targeting orthogonal openings in the intramedullary nail with the magnetic targeting device; and
o. drilling holes in the bone through the orthogonal openings.

33. The method of claim 26 wherein the aligning in step (d) further includes inducing a pulsed magnetic field by superimposing a fluctuating magnetic field upon a static magnetic field produced by the magnet member.

34. A bone plate targeting apparatus for targeting a bone plate including holes, the apparatus comprising:
a magnet member disposed a defined distance from at least one of the holes; and
a magnetic targeting device capable of detecting the magnet member including:
a support member having a proximal end and a distal end and having a drill sleeve extending therethrough;
a sensor array disposed on the distal end of the support member, wherein a distance between the sensor array and a lower opening of the drill guide corresponds with the defined distance; and
a positional indicator.

35. The apparatus of claim 34 wherein the magnet member is a ring magnet embedded around the at least one of the holes.

36. The apparatus of claim 35 wherein the magnet member threads into the at least one of the holes.

37. A method of targeting holes in a bone plate for the external fixation of a bone within a limb, the method comprising:
a. placing the bone plate against the bone, wherein the bone plate comprises a magnet member disposed a defined distance from at least one of the holes;
b. placing a magnetic targeting device against the bone plate, wherein the magnetic targeting device includes:
a support member having a proximal end and a distal end and having a drill sleeve extending therethrough;
a sensor array disposed on the distal end of the support member, wherein a distance between the sensor array and a lower opening of the drill guide corresponds with the defined distance; and
a positional indicator;
c. aligning the magnet member with the sensor array, wherein the aligning the magnet member with the sensor array aligns the lower opening of the drill guide with the at least one of the holes; and
d. drilling a hole in the bone through the at least one of the holes.
